(19) United States
(12) Patent Application Publication
(10) Pub. No.: US 2005/0284774 A1
(43) Pub. Date: Dec. 29, 2005

(54) Ophthalmic lens assembly utilizing replaceable contact element

(76) Inventor: David H. Mordaunt, Los Gatos, CA (US)

Correspondence Address: DLA PIPER RUDNICK GRAY CARY US, LLP, 2000 UNIVERSITY AVENUE, E. PALO ALTO, CA, 94303-2248, US

(21) Appl. No.: 11/166,660

(22) Filed: Jun. 23, 2005

Related U.S. Application Data
(60) Provisional application No. 60/582,445, filed on Jun. 24, 2004.

(51) Int. Cl. A45C 11/04; A61B 3/00

(52) U.S. Cl. 206/5.1; 351/219

(57) ABSTRACT

An ophthalmic lens assembly that includes a first lens mounted to a first housing and a second lens mounted to a second housing having an open end. The first housing at least partially inserts into the open end of the second housing and removably attaches to the second housing so that the first lens is fixed in a predefined alignment position relative to the second lens. The second lens and housing can be provided in a sterile sealed package. Alternately, the ophthalmic lens can include a lens assembly having first and second lenses mounted to a housing, and a transparent contact element removably attachable to the lens assembly for covering the second lens. The transparent contact element can be provided in a sterile sealed package.

This application claims the benefit of U.S. Provisional Application No. 60/582,445, filed Jun. 24, 2004.

### FIELD OF THE INVENTION

The present invention pertains generally to an ophthalmic contact lens with a replaceable patient contact element. The present invention is particularly useful as a ophthalmic lens with a packaged, sterile replaceable contact element.

### BACKGROUND OF THE INVENTION

There are a variety of ways in which to ophthalmoscopically observe the eye. Many of which involve the use an ophthalmic contact lens. Many such ophthalmic contact lenses require the use of an index-matching lubricant, such as hydroxypropyl methylcellulose, to minimize both the risk of scratching the patient's cornea, and spurious back-reflections from the cornea. To date, however, the design of such lenses has focused on their initial optical quality, and not on the more clinical concerns regarding their practicality, robustness, and role in the possible spread of infection associated with continuous use.

While optical quality is certainly important, the clinical use of these lenses is still awkward and time consuming for both patient and physician, as well as risky for the patient. This is because ophthalmic contact lenses are built (and priced) to be used continuously for as long as possible. One lens typically lasts about six months. Replacement is often due to damage to the contact element as a result of mishandling, as it is necessarily exposed and therefore vulnerable. During its lifetime, a lens will be typically used on numerous patients with only minimal cleaning in between. The lubricant, when used, is typically just wiped off after use, and then reapplied from the same bottle for the next patient. Although such lenses are designed for sterilization, the procedure is long and involved. For example, it is known that handling instructions for inter-patient lenses often include instructions such as: 1. Rinse: Immediately upon removal from a patient's eye, thoroughly rinse the lens in cool or tepid water. 2. Wash: Place a few drops of mild soap on a moistened cotton ball, and gently clean the entire lens using a circular motion. 3. Rinse: Thoroughly rinse in cool or tepid water, then dry carefully with a non-linting tissue. 4. Disinfect: First, soak the entire lens in a 2% or 3.4% aqueous solution of glutaraldehyde for a minimum exposure time of 20 minutes, or a 10% aqueous solution of chlorine bleach for 10 minutes. 5. Rinse: Thoroughly rinse the lens to remove any residual disinfectant, 3 cycles of 1 minute each with cool or tepid water. 6. EO Sterilize: Minimum exposure time of 1 hour with a temperature of 130 F, followed by 12 hours of aeration. Following this cleaning protocol removes the lens from further use for an entire workday. The demanding schedule of a practicing ophthalmologist typically disallows such delays. The obvious solution is to keep a multitude of lenses on hand. However, such ophthalmic lenses are fairly expensive, so this is costly.

In U.S. Pat. No. 3,820,879, Frisen discloses the use of multiple internal mirrors in order to provide for an upright image. Likewise, in U.S. Pat. No. 4,664,490, Rol discloses the use of internal mirrors to redirect light in order to see into the periphery of the eye's anterior chamber. While the use of internal mirrors incorporates significant clinical utility, it nonetheless disregards the practical requirements of robustness and sterility.

Heacock, et al. disclose the use of providing an aerial retinal image to improve the overall optical performance of ophthalmic contact lenses in U.S. Pat. No. 4,728,183. The ophthalmic lens disclosed therein includes contact lens and an aspheric entry lens to provide an aerial image of the fundus anterior to the entry lens.

In U.S. Pat. No. 6,373,571, Juhasz, et al. describe a system employing a disposable contact lens. It is designed for use in refractive surgery in order to properly align a lens and, thus, a laser system to a predetermined location by using reference marks made on the contact lens which are made to coincide with the laser beam itself. This is done specifically for use with an ultrafast laser system which is used to separate tissue, such as might be done when cutting a corneal flap prior to a LASIK procedure. Unlike the other approaches discussed above, the '571 patent does not address the optical quality of the ophthalmic lens. Rather, it concentrates on the precision of its optical alignment, without addressing the practical requirements of robustness and sterility.

There is a need for a device in this area that addresses the clinical requirements of robustness, sterility, and cost. Such a device would be useful not only to minimize the spread of infection, but also to ensure that the most crucial and inherently most vulnerable element of any ophthalmic lens is easily replaceable. Ideally, a new contact lens would be used for each patient. When packaged together with pre-applied lubricant, a completely sterile contact element can be used for each patient. Furthermore, rather than keep more than one complete lens on hand, a physician may simply have multiple contact elements, and thus reduce costs and improve efficiency without sacrificing either patient safety or comfort. Such a device lends itself to economical and straightforward manufacture, distribution, and ultimately, clinical use.

### SUMMARY OF THE INVENTION

The present invention is an ophthalmic lens assembly that includes a first housing, a first lens mounted to the first housing, a second housing having an open end, and a second lens mounted to the second housing, wherein the first housing at least partially inserts into the open end and removably attaches to the second housing so that the first lens is fixed in a predefined alignment position relative to the second lens.

In another aspect of the present invention, an ophthalmic lens includes a lens assembly having a housing and a first lens mounted to the housing and a second lens mounted to the housing, and a transparent contact element removably attachable to the lens assembly for covering the second lens.

In yet another aspect of the present invention, a method of preparing an ophthalmic lens assembly includes providing a first lens mounted to a first housing, providing a sterilized contact lens assembly in a sealed package, wherein the contact lens assembly includes a second lens mounted to a second housing, and wherein the second housing includes an open end, opening the sealed package and removing the contact lens assembly from the package, and inserting at least a portion of the first housing into the open end to removably attach the first and second housings together so that the first lens is fixed in a predefined alignment position relative to the second lens.

In yet one more aspect of the invention, a method of preparing an ophthalmic lens assembly includes providing an ophthalmic lens assembly having a housing on which first and second lenses are mounted, providing a sterilized transparent contact element in a sealed package, opening the sealed package and removing the contact element from the package, and placing the contact element over the second lens to removably attach the contact element to the ophthalmic lens assembly such that the contact element covers the second lens.

Other objects and features of the present invention will become apparent by a review of the specification, claims and appended figures.

### BRIEF DESCRIPTION OF THE DRAWINGS

FIG. 1 is a side cross-sectional view of the ophthalmic lens assembly of the present invention.

FIG. 2 is a top schematic representation of the ophthalmic lens of the present invention in a sterile package.

FIG. 3 is a side cross-sectional view an alternate embodiment of the present invention.

FIG. 4 is a top schematic representation of the contact element of the alternate embodiment of the present invention in a sterile package.

### DETAILED DESCRIPTION OF THE PREFERRED EMBODIMENTS

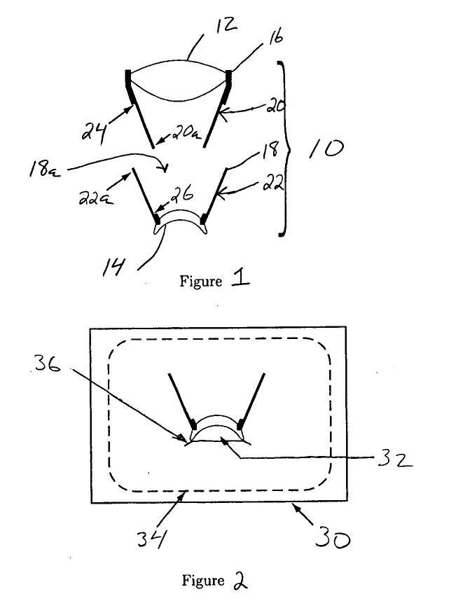

The present invention is an ophthalmic lens assembly 10 as illustrated in FIG. 1, which provides a means for an economical, easily replaceable, and sterile solution to the problems associated with the repeated use of ophthalmic contact lenses. The ophthalmic lens assembly 10 includes a primary lens 12 and a contact lens 14. Primary lens 12 is mounted to a first (primary) housing 16 and contact lens 14 is mounted to a second (contact) housing 18. Contact housing 18 may be made of a single molded piece, or an assembly of pieces, and with material(s) suitable for sterilization at least once (e.g. metal, plastic, etc.).

It should be noted that the term “lens” as used herein includes optical elements through which light passes having, or not having, any optical (focusing) power. Likewise, any lens may be formed of one or more optical elements.

Each housing 16 and 18 includes mating segments 20 and 22, respectively, for attaching housings 16/18 together. Specifically, housing 18 has an open end 18a in which housing 16 at least partially inserts (i.e. so that segment end 22a abuts shoulder 24 on mating segment 20, and segment end 20a abuts shoulder 26 on mating segment 22). In this manner, housing 18 covers a considerable portion of the exterior of housing 16. Thus, the interface between housings 16/18 (segment end 22a and shoulder 24) is located away from contact lens 14, creating a shroud around substantially the entire ophthalmic lens assembly 10. This configuration better ensures that a sterile field is maintained around contact lens 14, making it easier for the user to handle the assembly 10 well away from contact lens 14.

Housings 16/18 are removably attached together using any appropriate fixation scheme, such as threads, bayonet mounting, twist-lock, plunger & detent, slip-fitting, o-rings, etc. Once assembled, the internal surfaces of these components are removed from possible patient contact, and are thus of no concern for sterility. Furthermore, contact housing 18 may be packaged with lubricant gel applied to the distal surface of contact lens 14 to insure sterility. It should be noted that additional optical elements could be incorporated in the lens assembly 10, so long as a sterile field is maintained on those exposed surfaces nearest the contact lens 14 (and thus the patient).

With the configuration described above and illustrated in FIG. 1, only the contact housing 18 and contact lens 14 need be sterilized. Primary lens 12 and housing 16, being mostly contained within contact housing 18, are sufficiently separated from exposed surfaces of assembly 10 near the patient that further sterilization is not necessary. Therefore, contact housing 18 (with lens 14 mounted thereon) can be packaged in sterile packaging 30 (with lubricant gel 32 already applied to the patient contact side of contact lens 14, if desired), and sealed by seal 34 for sterile distribution. Such packaging may be a blister pack, as is commonly employed in clinical environments, although other possibilities exist. When lubricant gel 32 is pre-applied to contact lens 14, it may be contained (and protected from potential handling) by an optional cover 36. Cover 36 would be removed just prior to use, and thus the patient contact surface of the contact lens 14 remains sterile. In the case where contact housing 18 and lens 14 are repeatedly sterilizable, cover 36 and package 30 may be also made to be reused (e.g. from metal, plastic, etc.). This way they can serve as safe and clean storage for contact housing/lens 18/14. Alternatively, contact housing/lens 18/14 can be made of disposable materials, so that they can be disposed of after use.

The present invention provides a tremendous advantage over the prior art. By having the contact lens removably attachable to the primary lens, it is far more cost effective to supply pre-sterilized contact lenses for attachment primary lenses, and even makes it cost effective to supply the contact lens and its housing in a disposable configuration. Moreover, there is no need to sterilize the primary lens, especially where the contact lens housing surrounds and covers most of the primary lens housing. The primary and contact lenses are reliably secured together in a predefined alignment relationship without contaminating either the contact lens or the exposed surfaces of the contact lens housing adjacent thereto.

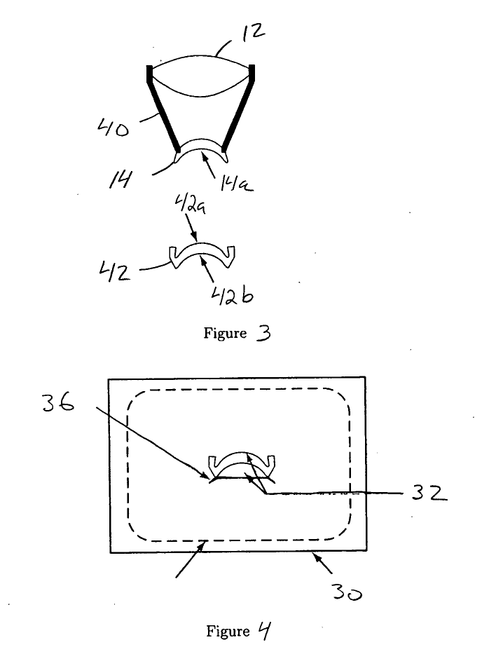

FIG. 3 illustrates an alternate embodiment of the present invention, in which primary and contact lenses 12/14 are both mounted to a unitary housing 40. Sterility is provided by an inexpensive and replaceable contact element 42, which may be readily discarded after use. Contact element 42 may be made of a material that is soft, transparent, sterilizable, and biocompatible, such as silicone. Replaceable contact element 42 is made to be sterilized, and mounts to contact lens 14 and/or housing 40. Contact element 42 preferably has a lens contact surface 42a that matches the curvature of the contact lens surface 14a to which it mounts. Contact element 42 also includes a patient contact surface 42b that preferably matches the curvature of contact lens surface 14a, so that the optical power of contact element 42 is minimized and will not substantially perturb the optical performance of lenses 12/14.

It should be noted that even if contact element 42 is made to have no inherent optical power, it still may change the optical performance of lenses 12/14. The amount and type of such changes, although typically small, is highly dependent upon the specific lens itself. As an example, a contact element 42 made of acrylic and having a thickness of 0.5 mm can increase the effective focal length by about 0.25 mm, and can slightly increase the magnitude of certain optical aberrations. To ameliorate these possible effects, the design of lenses 12/14 may be optimized to counteract the effects of contact element 42. Furthermore, an index-matching lubricant gel may be placed at the interface between surfaces 14a and 42a to minimize any reflections at this interface. Contact element 42 may be packaged with lubricant gel 32 on surface 42a for this purpose, as well as on surface 42b for patient comfort and safety. FIG. 4 illustrates contact element 42 packaged in sterile packaging 30, with lubricant gel 32 already applied to the both surfaces 42a and 42b (with cover 36 maintaining the gel on surface 42b).

It is to be understood that the present invention is not limited to the embodiment(s) described above and illustrated herein, but encompasses any and all variations falling within the scope of the appended claims.

1. An ophthalmic lens assembly, comprising:
a first housing;
a first lens mounted to the first housing;
a second housing having an open end;
a second lens mounted to the second housing;
wherein the first housing at least partially inserts into the open end and removably attaches to the second housing so that the first lens is fixed in a predefined alignment position relative to the second lens.

2. The ophthalmic lens assembly of claim 1, wherein:
the first housing includes a first mating segment;
the second housing includes a second mating segment; and
as the first housing inserts into the open end, an outer surface of the first mating segment engages an inner surface of the second mating segment so that the second mating segment covers the first mating segment.

3. The ophthalmic lens assembly of claim 1, wherein:
the first housing includes a first mating segment having a first shoulder and a first end;
the second housing includes a second mating segment having a second shoulder and a second end; and
as the first housing inserts into the open end, the first and second mating segments mate together by the first end abutting the second shoulder and the second end abutting the first shoulder.

4. The ophthalmic lens assembly of claim 1, wherein the first housing has an outer surface, and wherein a portion of the outer surface is covered by the second housing as the first housing inserts into the open end.

5. The ophthalmic lens assembly of claim 1, wherein the second housing includes a distal end opposite the open end, and wherein the second lens is mounted to the second housing at the distal end.

6. The ophthalmic lens assembly of claim 1, further comprising:
a lubricant gel disposed on the second lens.

7. The ophthalmic lens assembly of claim 6, further comprising:
a cover member disposed on the second lens and over the lubricant gel.

8. An ophthalmic lens, comprising:
a lens assembly including:
a housing,
a first lens mounted to the housing, and
a second lens mounted to the housing; and
a transparent contact element removably attachable to the lens assembly for covering the second lens.

9. The ophthalmic lens of claim 8, wherein the contact element includes a first surface facing the second lens that has a curvature matching that of a distal surface of the second lens.

10. The ophthalmic lens of claim 9, wherein the contact element includes a second surface having a curvature matching that of the first surface.

11. The ophthalmic lens of claim 8, further comprising:
a lubricant gel disposed on the contact element.

12. The ophthalmic lens of claim 11, further comprising:
a cover member disposed on the contact element and over the lubricant gel.

13. A method of preparing an ophthalmic lens assembly, comprising:
providing a first lens mounted to a first housing;
providing a sterilized contact lens assembly in a sealed package, wherein the contact lens assembly includes a second lens mounted to a second housing, and wherein the second housing includes an open end;
opening the sealed package and removing the contact lens assembly from the package; and
inserting at least a portion of the first housing into the open end to removably attach the first and second housings together so that the first lens is fixed in a predefined alignment position relative to the second lens.

14. The method of claim 13, wherein the first housing includes a first mating segment and the second housing includes a second mating segment, and wherein as the first housing is inserted into the open end, an outer surface of the first mating segment engages an inner surface of the second mating segment so that the second mating segment covers the first mating segment.

15. The method of claim 13, wherein:
the first housing includes a first mating segment having a first shoulder and a first end;
the second housing includes a second mating segment having a second shoulder and a second end; and
as the first housing is inserted into the open end, the first and second mating segments mate together by the first end abutting the second shoulder and the second end abutting the first shoulder.

16. The method of claim 13, wherein the first housing has an outer surface, and wherein a portion of the outer surface is covered by the second housing as the first housing is inserted into the open end.

17. The method of claim 13, further comprising:
providing a lubricant gel on the second lens.

18. The method of claim 17, further comprising:
providing a cover member on the second lens and over the lubricant gel.

19. A method of preparing an ophthalmic lens assembly, comprising:
providing an ophthalmic lens assembly having a housing on which first and second lenses are mounted;
providing a sterilized transparent contact element in a sealed package;
opening the sealed package and removing the contact element from the package; and
placing the contact element over the second lens to removably attach the contact element to the ophthalmic lens assembly such that the contact element covers the second lens.

20. The method of claim 19, wherein the contact element includes a first surface facing the second lens that has a curvature matching that of a distal surface of the second lens.

21. The method of claim 20, wherein the contact element includes a second surface having a curvature matching that of the first surface.

22. The method of claim 19, further comprising:
providing a lubricant gel on the contact element.

23. The method of claim 22, further comprising:
providing a cover member on the contact element and over the lubricant gel.
